(19) United States
(12) Patent Application Publication
(10) Pub. No.: US 2013/0274730 A1
(43) Pub. Date: Oct. 17, 2013

(54) ABLATION CATHETER AND METHODS FOR NERVE MODULATION

(71) Applicant: BOSTON SCIENTIFIC SCIMED, INC., MAPLE GROVE, MN (US)

(72) Inventors: JAMES M. ANDERSON, FRIDLEY, MN (US); HUISUN WANG, MAPLE GROVE, MN (US); RICHARD C. GUNDERSON, MAPLE GROVE, MN (US)

(73) Assignee: BOSTON SCIENTIFIC SCIMED, INC., MAPLE GROVE, MN (US)

(21) Appl. No.: 13/860,414

(22) Filed: Apr. 10, 2013

Related U.S. Application Data
(60) Provisional application No. 61/623,369, filed on Apr. 12, 2012.

(51) Int. Cl. A61B 18/18

(52) U.S. Cl. CPC A61B 18/18
USPC 606/33

(57) ABSTRACT

Ablative catheters and methods for making and using the same are disclosed. The catheter may have a proximal end, a distal end, and a first lumen extending at least partially between the proximal and distal ends. The catheter may include at least one ablative port located adjacent to the distal end of the catheter. The catheter may include an ablative mechanism capable of ablation using an conductive fluid circulated to the ablative port through the first lumen. An expandable member may be disposed at an outer surface of a distal portion of the catheter. The expandable member may be capable of switching between a collapsed position and an expanded position using an expansion fluid circulated to the expandable member through a second lumen. In the expanded position, the expandable member may be sized and shaped to position the ablative port at a suitable distance from a wall of the vessel.

### CROSS-REFERENCE TO RELATED APPLICATIONS

This application claims priority under 35 U.S.C. §119 to U.S. Provisional Application Ser. No. 61/623,369, filed Apr. 12, 2012, the entirety of which is incorporated herein by reference.

### TECHNICAL FIELD

The present disclosure relates to methods and apparatuses for modulating nerves through the walls of blood vessels. Such modulation may include ablation of nerve tissue or other nerve modulation techniques.

### BACKGROUND

Certain treatments utilize temporary or permanent interruption or modification of select nerve functions. One example treatment is renal nerve ablation, which is sometimes used to treat conditions related to congestive heart failure. The kidneys produce a sympathetic response to congestive heart failure, which among other effects, increases the undesired retention of water and/or sodium. Ablating some nerves associated with the kidneys may reduce or eliminate this sympathetic function, providing a corresponding reduction in the associated undesired effects.

Many nerves (and nerve tissue such as brain tissue), including renal nerves, run along the walls of or in close proximity to blood vessels and these nerves can be accessed intravascularly through the blood vessel walls. In some instances, it may be desirable to ablate perivascular renal nerves using a radio frequency (RF) electrode. Such treatment, however, may result in thermal injury to the vessel from the electrode and other undesirable side effects such as, but not limited to, blood damage, clotting, and/or protein fouling of the electrode. To prevent such undesirable side effects, some techniques attempt to increase the distance between the vessel walls and the electrode. In these systems, however, the electrode may inadvertently contact the vessel walls, causing irreparable damage.

Therefore, there remains a need for improvement and/or alternatives in providing systems and methods for intravascular nerve modulation.

### SUMMARY

The disclosure is directed to several alternative designs, materials, and methods of manufacturing medical device structures and assemblies.

Accordingly, some embodiments pertain to an ablative catheter system for nerve modulation through the wall of a blood vessel. The catheter system includes a catheter having a proximal end, a distal end, and at least two lumens extending at least partially between the proximal and distal ends. In addition, the catheter includes at least one ablative port located proximate to the distal end of the catheter. Further, the catheter is rotatable about its elongate axis. An ablative mechanism capable of ablation using a conductive fluid that is circulated to the ablative port through one of the lumen. The lumen extends between the ablative port and the proximal end of the catheter. The conductive fluid is an electrically conductive fluid capable of transferring radio frequency current from a current source to a target area. In an illustrated embodiment, saline is used as a conductive fluid to provide an electrical path for radio frequency energy to travel from the current source to the vessel wall. Further, an expandable member is disposed at the outer surface of the distal portion of the catheter. The expandable member may be capable of switching between a collapsed position and an expanded position using an expansion fluid circulated to the expandable member through the second lumen. In the expanded position, the expandable member is sized and shaped to position the ablative port at a suitable distance from the vessel wall. In an illustrated embodiment, the expandable member is an asymmetrically shaped balloon. The expandable member is either self-expandable or expanded by an actuating means. The second lumen extends between the proximal end of the catheter and an opening in the expandable member. In another embodiment, a single lumen is used to inflate the expandable member and provide the conductive fluid for the ablation procedure. The fluid is provided to the expandable member and then from the expandable member to the ablation port.

Some other embodiments pertain to a renal ablation system for nerve modulation through the wall of a blood vessel. The renal ablation system includes an elongate member having a proximal end, a distal end, and at least one ablative port located proximate to the distal end of the elongate member. In addition, the elongate member is rotatable about its elongate axis. An ablative mechanism capable of ablation by using saline as a conductive medium to prove a path between the radio frequency energy source and the targeted portions of the vessel wall. An expandable member is disposed at the outer surface of a distal portion of the catheter. The expandable member may be capable of moving between a collapsed position and an expanded position using an expansion fluid circulated to the expandable member through the second lumen. In expanded position, the expandable member is sized and shaped to position the ablative port is at a distance from the vessel wall. In the illustrated embodiments, the expandable member is an asymmetrically shaped balloon and the expansion fluid is an inflation fluid.

Some instances also pertain to a method for ablating a nerve perivascularly through a vessel lumen where an ablative catheter is advanced intravascularly proximate a desired location in the vessel lumen. The ablative catheter includes a proximal end, a distal end, an ablative port disposed at a location proximate the distal end of the ablative catheter, and expandable member disposed at the outer surface of a distal portion of the ablative catheter. The expandable member may then be deployed in an expanded position in the vessel lumen such that portions of the expandable member contact the walls of the vessel lumen and the ablative port maintains a distance from the walls of the vessel lumen. While an electrode at the ablation port is activated, saline may then be ejected through the port and directed towards the vessel wall.

The summary of some example embodiments in not intended to describe each disclosed embodiment or every implementation of the disclosure.

### BRIEF DESCRIPTION OF THE DRAWINGS

The present disclosure may be more completely understood in consideration of the following detailed description of various embodiments in connection with the accompanying drawings, in which:

FIG. 1 is a schematic view of an illustrative renal nerve modulation system in situ, of the present disclosure.

FIG. 2 illustrates an exemplary ablative catheter system of the present disclosure.

FIGS. 3A and 3B illustrate a cross-sectional view of a renal artery including the exemplary ablative catheter system of FIG. 2 having an expandable member in an expanded position and a collapsed position, respectively.

FIGS. 4A, 4B, 4C, and 4D cross-sectional view illustrating multiple orientations of the renal artery with the exemplary ablative catheter system of FIG. 3A, operatively positioned therein.

FIGS. 5A, 5B, 5C, and 5D illustrate alternate exemplary embodiments of the expandable member, illustrated in FIG. 2.

FIGS. 6A, 6B, and 6C are perspective views of multiple exemplary embodiments of the ablative catheter system illustrating different ablative ports of the present disclosure.

FIG. 7 is cross-sectional side view of another exemplary ablative catheter system of the present disclosure.

While embodiments of the present disclosure are amenable to various modifications and alternative forms, specifics thereof have been shown by way of example in the drawings and will be described in detail. It should be understood, however, that the intention is not to limit aspects of the disclosure to the particular embodiments described. One the contrary, the intention is to cover all modifications, equivalents, and alternatives falling within the spirit and scope of the present disclosure.

### DETAILED DESCRIPTION

For the following defined terms, these definitions shall be applied, unless a different definition is provided in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant numbers.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. For example, the device and methods may be used in any other blood vessels or body lumen where nerve modulation or other tissue modulation is desired.

In some instances, it may be desirable to ablate perivascular renal nerves with deep target tissue heating. However, as energy passes from an electrode to the desired treatment region, the energy may heat the fluid (e.g. blood) and tissue as it passes. As more energy is used, higher temperatures in the desired treatment region may be achieved, but may result in some negative side effects, such as, but not limited to, thermal injury to the blood vessel wall, damage to or clotting of the blood cells themselves, and/or fouling the electrode. Positioning the electrode away from the blood vessel wall may provide some degree of passive cooling by allowing blood to flow past the electrode. Further, in embodiments described herein, the electrical energy providing the ablation treatment is provided through the vessel wall by a fluid. This fluid may provide further cooling to the vessel wall.

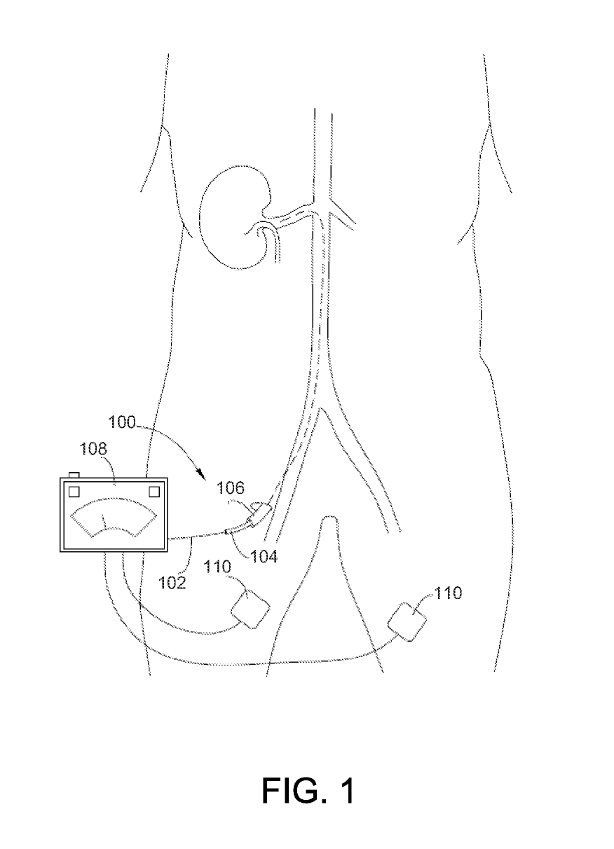

FIG. 1 is a schematic view of an illustrative renal nerve modulation system 100 in situ. System 100 may include one or more conductors 102 for providing power to an electrical conductor 104 disposed within a catheter 106, the details of which can be better seen in subsequent figures. A proximal end of the conductor 102 may be connected to a control and power element 108, which supplies the necessary electrical energy to activate the one or more electrodes (not shown) at or near a distal end of the electrical conductor 104. In some instances, return electrode patches 110 may be supplied on the legs, abdomen, or at another conventional location on the patient's body to complete the circuit. The control and power element 108 may include monitoring elements to monitor parameters such as power, temperature, voltage, pulse size and/or shape and other suitable parameters as well as suitable controls for performing the desired procedure. In some instances, the power element 108 may control a radio frequency (RF) electrode. The electrode may be capable of operating at a frequency of about 460 kHz. It is contemplated that any desired frequency in the RF range may be used, for example, from about 450 to about 500 kHz. However, it is also contemplated that different types of energy outside the RF spectrum may be used as desired, such as, but not limited to, ultrasound, microwave, and laser. Other elements may include an introducer sheath and/or a guidewire. The system may also include a source of inflation fluid and/or a source of conductive fluid. These may be attached to the catheter using a conventional catheter hub with luer ports or the like. The fluid sources may be syringes or may include electronically controlled pumps to control the rate or volume of fluid introduced.

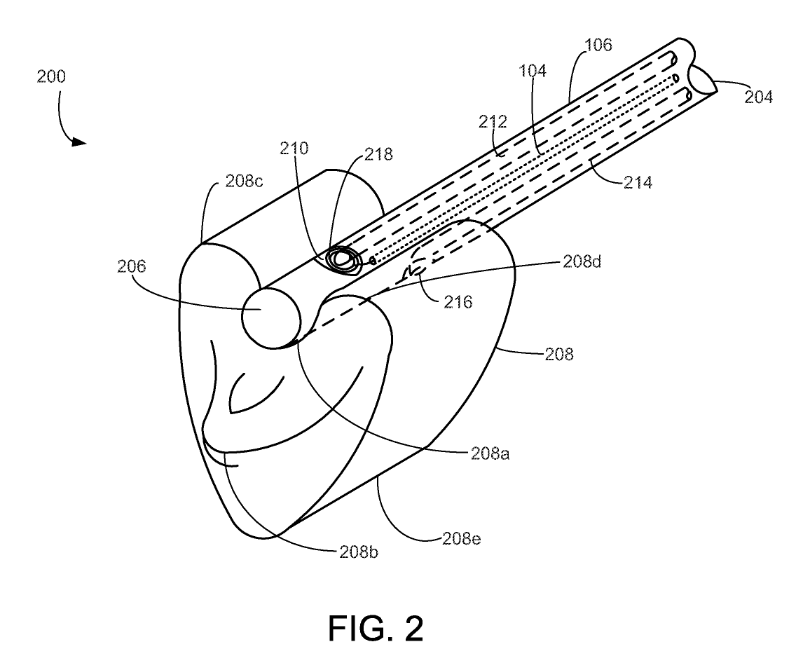

FIG. 2 illustrates an exemplary ablative catheter system 200 according to embodiments of the present disclosure. The ablative catheter system 200 includes the catheter 106 having a proximal end 204, a distal end 206, and lumen 212 and 214 extending at least partially between the proximal and distal ends 204, 206. A location proximate the distal end 206 of catheter 106 includes an ablative port 210 for ablative purposes. In addition, an expandable member 208 is disposed partially surrounding the outer surface of a distal portion of the catheter 106.

The catheter 106 may be a substantially circular hollow tube like structure. Other suitable cross-sectional shapes of the catheter 106 may be elliptical, oval, polygonal, or irregular. Moreover, the catheter 106 may be flexible along its entire length or adapted for flexure along portions of its length. Alternatively, the distal end 206 may be flexible while the more proximal portions of catheter 106 may be relatively rigid. During use, flexibility allows the catheter 106 to maneuver turns in the circuitous vasculature, while rigidity provides the necessary columnar strength to urge the catheter 106 forward to the intended target area. The diameter of the catheter 106 may vary according to the desired application, but it is generally smaller than the typical diameter of a patient's vasculature. A typical system, for example, may be compatible with a 6 French introducer sheath. The length of the catheter 106 may vary according to the location of the body lumen where the ablative process is to be conducted.

As shown, the distal end 206 of the catheter 106 may be closed while the proximal end 204 may include ports or openings (not shown) to insert desired devices within the catheter 106. The distal end 206 may be designed to reduce trauma and irritation to surrounding tissues. For example, the distal end 206 may include a rounded or beveled tip.

Catheter 106 may be made of one or more of any suitable biocompatible material such as a polymeric, or any other such material for example, a polymeric, electrically nonconductive material, such as polyethylene, polyurethane, or PEBAX® material (polyurethane and nylon) or a metal such as stainless steel or a nickel-titanium alloy. In some instances, the catheter 106 may include a portion having a wire braid imbedded in a polymer material to impart flexibility. In addition, the distal end 206 may be made more flexible than the proximal portion by using different material and/or having a thinner wall thickness. Varying the flexibility of the material may have the benefit of reducing the risk of injury to blood vessel walls, which the distal end 206 may contact, during a medical procedure.

Ablative port 210 may be one or more openings in the distal portion of the wall of the catheter 106. As illustrated, the ablative port 210 may be connected to the interior lumen 212, and the ablative port 210 along with lumen 212 may provide perivascular nerve ablation using an conductive fluid. The ablative port 210 may eject a stream of conductive fluid to the wall of a blood vessel for ablation.

The shape and size of the ablative port 210 depends upon the desired rate of flow of the conductive fluid from the ablative port 210 and the area of the vessel wall that needs to be ablated. For example, the ablative port 210 may be a single opening with a nozzle to produce a steady stream of conductive fluid. Alternatively, the ablative port 210 may be a group of small openings spread out in an area on the surface of the catheter 106 to produce a lower intensity stream of conductive fluid. It should be understood that many variations between a high velocity and a low velocity stream are contemplated by the present disclosure. A suitable flow rate may be between 2 ml/min and 20 ml/min. In some embodiments, the fluid should be ejected from the ablative port such that the force of the fluid against the vessel wall does not damage the vessel wall. The appropriate flow rate may be dependent on the size of ablative port 210.

The conductive fluid ablates the vessel wall by transferring radio frequency electrical current from an electrode 218 present at the distal end of the electrical conductor 104 to the vessel wall. In general, the conductive fluid acts as a conducting medium to transfer radio frequency electrical current. The conductive fluid may generally be a water soluble, biocompatible, non-toxic, and electrically conductive fluid. Suitable fluids that may be used as the conductive fluid include salines such as isotonic saline and the like. In addition, a quantity of radiopaque fluid may be used as well. The radiopaque fluid may be mixed with the conductive fluid to provide for constant visualization or may be introduced periodically and discretely through the fluid channels to provide for visualization at discrete intervals.

The electrode 218 may be brought in electrical contact with the conductive fluid using any suitable mechanism. As shown, the electrical conductor 104 may be disposed exterior to the lumen 212 within the catheter 106 and the radio frequency electrode 218 may connect to the ablative port 210. Alternatively, the electrical conductor 104 may extend through lumen 212.

Expandable member 208 may be disposed on or adjacent to the catheter 106 proximate to the distal end 206 such that it covers a sufficient outer surface of the catheter 106 to expose the ablative port 210 so that the conductive fluid may contact the target area. The expandable member 208 may be any apparatus that may expand upon actuation such as an endoscopic basket, balloons, or any other mechanical device that can expand or the like. In the illustrated embodiment of the present disclosure, the expandable member 208 is an inflatable balloon that may shift between expanded and collapsed positions upon actuation. In addition, the expandable member 208 may be connected to the interior lumen 214 through a port 216.

As shown, the expandable member 208 may assume a configuration substantially similar to a cardioid in the plane perpendicular to the elongate axis of the catheter 106. The expandable member 208 includes a groove 208a, a front protrusion 208b, and side lobes 208c, 208d, and 208e.

The groove 208a may be located at the cusp of the cardioid. The catheter 106 may be attached to the expandable member 208 on the groove 208a. The groove 208a may have a width and depth greater than the diameter of the catheter 106 to accommodate the catheter 106.

The front protrusion 208b may be located at the distal surface of the expandable member 208 and extending beyond the distal end 206. The front protrusion 208b may act as a blunt atraumatic tip to prevent the tissue in contact with the distal end of the expandable member 208 from an inadvertent injury.

The side lobes 208c and 208d may be identical lobes located on the sides of the groove 208a. As illustrated, the ablative port 210 may be disposed on the catheter 106 between the side lobes 208c and 208d. The side lobes 208c and 208d may restrict the ablative port 210 from contacting the wall of a blood vessel. The dimensions of the side lobes 208c and 208d may determine the minimum distance of the ablative port 210 from the vessel wall. For example, long side lobes 208c and 208d may lead to larger distance between the ablative port 210 and the vessel wall while short side lobes 208c and 208d may lead to a shorter distance between the ablative port 210 and the vessel wall. Thus, the side lobes 208c and 208d of the expandable member 208 ensure that the ablative port 210 remains at a distance from vessel wall.

The lobe 208e may be substantially larger than the side lobes 208c and 208d and may be located in a direction opposite to the groove 208a and side lobes 208c and 208d. The lobe 208e along with the side lobes 208c and 208d may contact the vessel wall and facilitate in positioning the ablative catheter system 200 within the blood vessel.

In addition, the expandable member 208 may extend to a length along the elongate axis of the catheter 106. In general, the length of the expandable member 208 may be longer than the width of the ablative port 210.

The expandable member 208 may be connected to the surface of the catheter 106 using any sufficient attachment mechanisms. Some exemplary attachment mechanism may include adhesives or thermal bonding. For example, adhesives such as biocompatible resins or glue may be used to attach the expandable member 208 to the catheter 106.

The expandable member 208 may be made of any suitable biocompatible material such as polymers and rubbers. The expandable member 208 may be made from a compliant or a non-compliant material. The catheter 106 and the expandable member 208 may include suitable coatings on one or more of the surfaces. For example, the catheter 106 and expandable member 208 may be coated with suitable low friction material, such as TEFLON®, polyetheretherketone (PEEK), polyimide, nylon, polyethylene, or other lubricious polymer coatings, to reduce surface friction with the surrounding body tissues.

As discussed, the expandable member 208 may assume two configurations expanded and collapsed. These two configurations facilitate in the functioning of the ablative catheter system 200, and are discussed further in the following section.

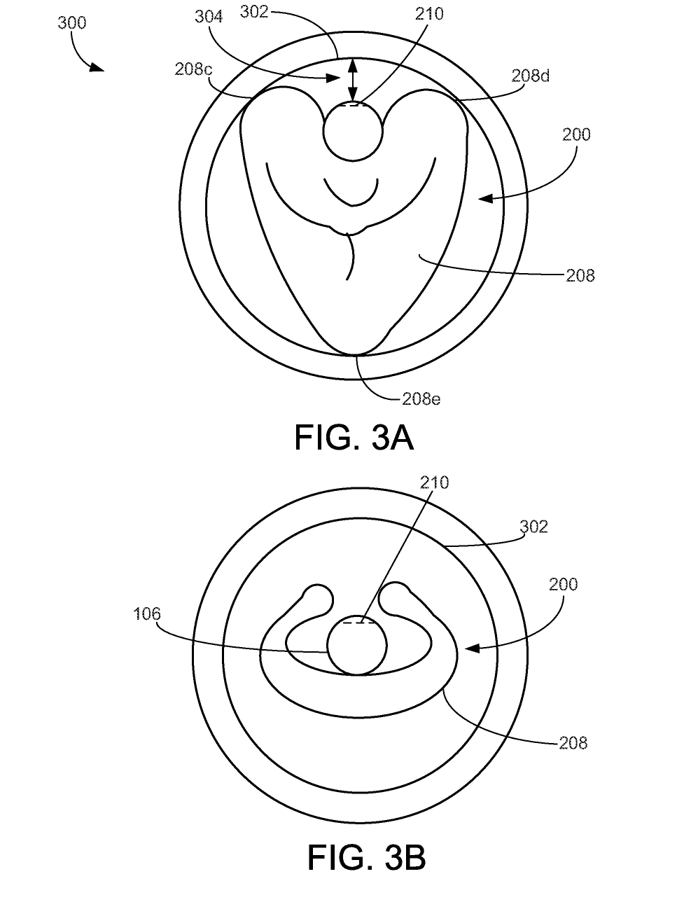

FIGS. 3A and 3B illustrate a cross-sectional view of a renal artery 300 including the ablative catheter system 200 having an expandable member 208 in its expanded position and collapsed position, respectively.

FIG. 3A exhibits the expandable member 208 in expanded position within the renal artery 300. The expandable member 208 in expanded position may assume the configuration illustrated in FIG. 2. The expandable member 208 may contact and exert force on the artery wall 302, which may expand the ablative catheter system 200 to a stable position within the renal artery 300.

A channel 304 may form between the inner artery wall 302 and the ablative catheter system 200. The channel 304 may have dimensions defined by the size of the expandable member 208 and the catheter 106, for example, the channel 304 may be as long as the length of the expandable member 208 along the elongate axis of the catheter 106. The channel 304 may have a width greater than the width of the catheter 106 and the depth of the channel 304 may be equal to the distance of the catheter 106 from the artery wall 302.

FIG. 3B exhibits the expandable member 208 in the collapsed position. In the collapsed position, the expandable member 208 may be wrapped around the outer surface of the catheter 106. Alternatively, the expandable member 208 may be disposed within a lumen in the catheter 106 and extend out of a port when expanded.

In the collapsed position, the size of the expandable member 208 may be considerably smaller than the diameter of the lumen of the renal artery 300. This small size of the expandable member 208 in its collapsed position may allow it to be slide easily within the lumen of the renal artery 300, and thus facilitate an operator to position the catheter 106 to any desired location within the renal artery 300. In addition, reduced size of the expandable member 208 may allow the catheter 106 to rotate easily within the renal artery 300.

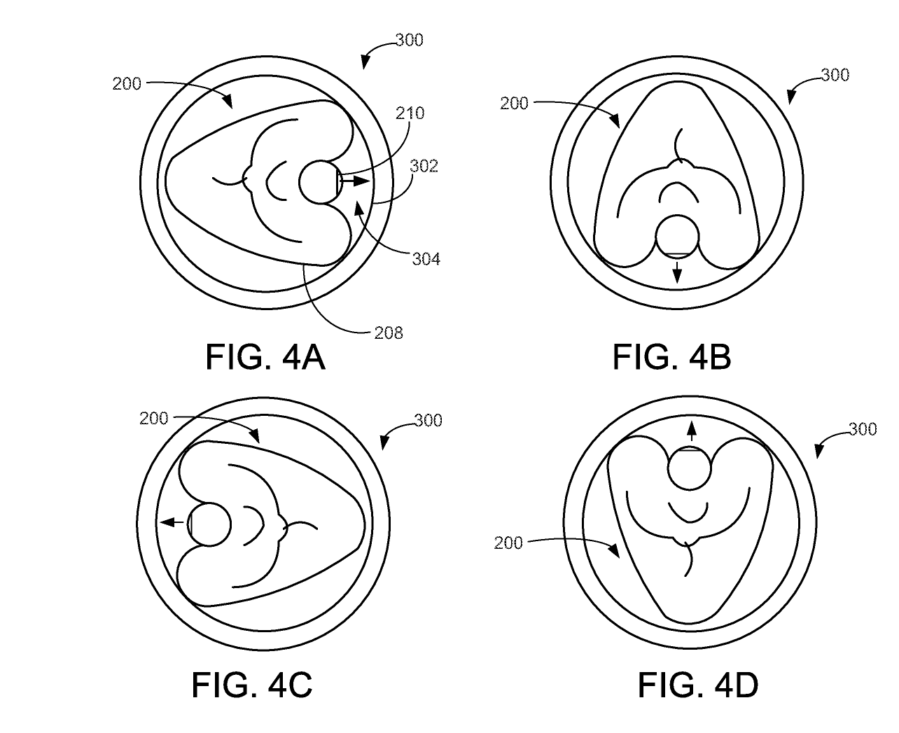

FIGS. 4A, 4B, 4C, and 4D illustrate cross-sectional views of the multiple orientations with the ablative catheter system 200 of FIG. 3A positioned in a renal artery 300. As discussed, the catheter 106 may be rotatable about the elongate axis within the renal artery 300. The rotatable property of the catheter 106 may allow the ablative catheter system 200 to be positioned in multiple orientations about the elongate axis, and ablate a large region of the vessel wall. In addition, rotatable catheter 106 may be easy to maneuver within a patient's circuitous vasculature.

In operation, an expansion mechanism may translate the expandable member 208 between expanded and collapsed positions while an ablative mechanism may ablate nerve tissue on vessel walls. The expansion mechanism and the ablative mechanism are described in detail the following sections.

The expansion mechanism may be any suitable mechanism that may translate the expandable member 208 between its expanded and collapsed positions upon actuation. The expansion mechanism may include use of inflation fluids, shape memory alloys, or other suitable mechanisms. In the illustrated embodiment of the present disclosure, the expansion mechanism includes use of an inflation fluid to translate the expandable member 208 between the expanded and collapsed positions. The inflation fluids may be any other biocompatible fluid such as isotonic saline. As shown in FIG. 2, the lumen 214 of the catheter 106 may provide the expandable member 208 with the inflation fluid.

In addition, the expansion mechanism may include other components, such as, a pressure source (not shown), a controller (not shown), and a fluid storage device (not shown). The inflation fluid, for example, saline may be stored in the fluid reservoir that may be connected to the expandable member 208 through lumen 214 and port 216. The fluid reservoir may be present inside or outside the ablative catheter system 200. The fluid reservoir may be a fluid cylinder, tank, or any other fluid storage device. The pressure source may apply pressure to expand or collapse the expandable member 208 by transferring fluid from the fluid reservoir to the expandable member 208 or from the expandable member 208 to the fluid reservoir through lumen 214 and port 216. The pressure source may be any pressurizing device, such as, a mechanical pump, electrical pump, a syringe, or other suitable mechanisms. The controller may control the operation of the pressure source by activating, deactivating the pressure source, or controlling the flow rate and amount of inflation fluid to be filled in the expandable member 208. The controller may be an independent element or the controller may be a part of the control and power element 108.

Once the expandable member 208 assumes its desired position with in the blood vessel, the ablative mechanism may be activated. As discussed, the ablative mechanism may include the conductive fluid, the electrical conductor 104, the ablative port 210, and the lumen 212. In addition, the ablative mechanism may include other components such as a pressure source (not shown), a controller (not shown), and a fluid reservoir (not shown).

The conductive fluid, saline, may be stored in the fluid reservoir that may be connected to the ablative port 210 through lumen 212. The fluid reservoir may be present inside or outside the ablative catheter system 200. The fluid reservoir may be a fluid cylinder, tank, or any other fluid storage device. The pressure source may apply pressure to expel the conductive fluid out of the ablative port 210 by transferring fluid from the fluid reservoir to the lumen 212. The increased fluid in the lumen 212 may thrust the conductive fluid out of the ablative port 210. The pressure source may be any pressurizing device, such as, a mechanical pump, electrical pump, a syringe, or the like. The controller may control the operation of the pressure source by activating, deactivating the pressure source, or controlling the flow rate and amount of the conductive fluid. The controller may be an independent element or the controller may be a part of the control and power element 108.

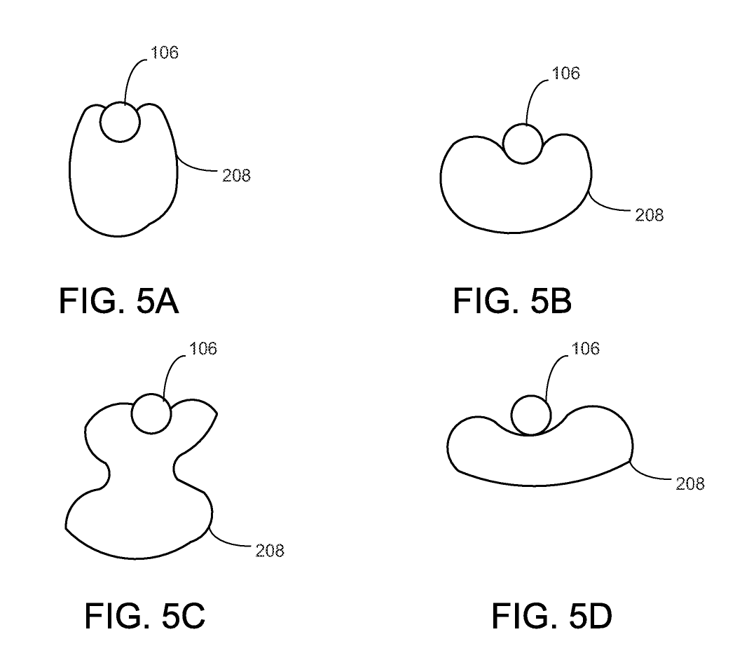

FIGS. 5A, 5B, 5C, and 5D illustrate multiple alternative embodiments of the ablative catheter system 200 with different contemplated shapes of the expandable member 208. It should be noted that the size and shape of the expandable member 208 described in the illustrated embodiments is merely exemplary and a person skilled in the art may find many other suitable configurations for the expandable member 208 that may be applicable for desired applications of the expandable member 208.

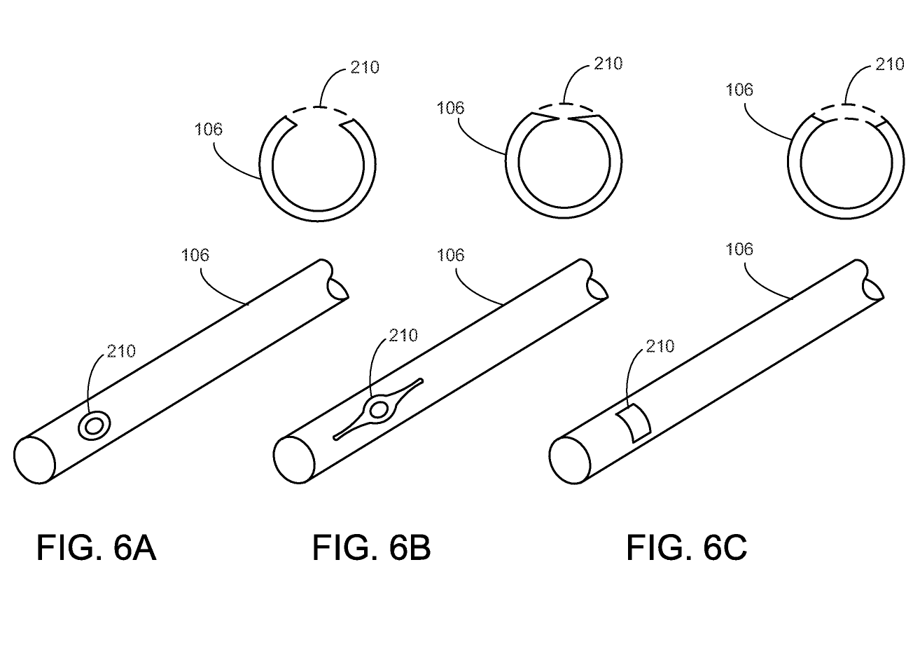

FIGS. 6A, 6B, and 6C illustrate multiple exemplary embodiments of the ablative catheter system 200 with different ablation ports. As discussed, the ablative port 210 may be designed with any suitable shape and suitable size based upon the desired application of the ablative catheter system 200 and the field of intended use. FIGS. 6A, 6B, and 6C illustrate three exemplary embodiments of the ablative catheter system 200, for the ablative port 210. FIG. 6A illustrates an embodiment of the ablative catheter system 200 with the ablative port 210 having a tapered structure. FIG. 6B exhibits an alternative embodiment of the ablative catheter system 200 with the ablative port 210 having a skived structure, and FIG. 6C describes another embodiment with oval slit structure of the ablative port 210. It may be noted that these embodiments of the ablative port 210 are only exemplary and a person skilled in the art may contemplate other suitable shaped and structures of the ablative port 210.

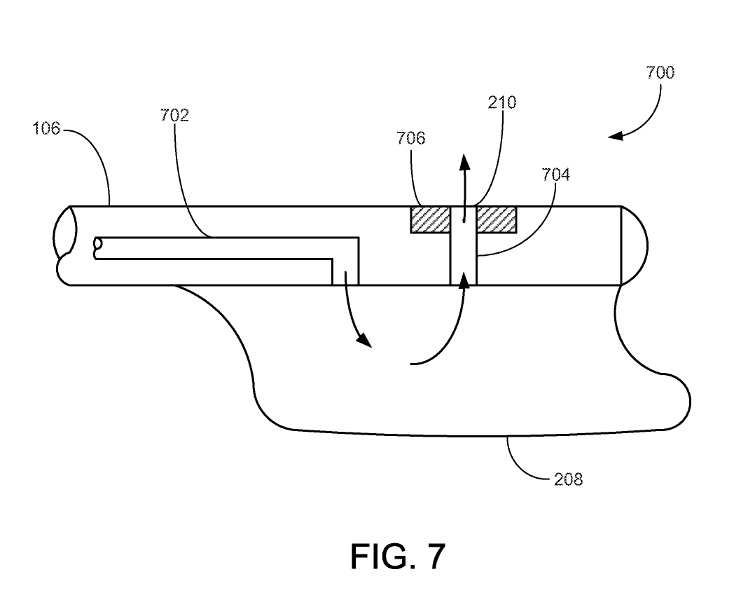

FIG. 7 is cross-sectional side view of a portion of another exemplary ablation catheter system 700. In this embodiment, a single conductive fluid may be used as both the inflation and conductive fluid. The embodiment illustrates that a single lumen 702 may inflate the expandable member 208 with the conductive fluid. Another lumen 704 may connect the expandable member 208 with the ablative port 210, and may transfer the conductive fluid from the expandable member 208 to the ablative port 210. A radio frequency electrode 706 disposed proximate the ablative port 210 may transfer radio frequency electrical energy to the conductive fluid in order to transfer thermal energy to the conductive fluid.

In operation, a pressure source (not shown) may transfer the conductive fluid from a fluid reservoir (not shown) to the lumen 702 at high pressure. The high-pressure conductive fluid may inflate the expandable member 208 and thrust out of lumen 704 from the expandable member 208. It may be desirable to keep the balloon at a pressure of between 1 and 5 atm or other pressure sufficient to keep the balloon in its expanded state. This balloon pressure is a function of the size of lumen 702, lumen 704, ablative port 210 and the flow rate. The inflow rate may be controlled by monitoring the balloon pressure. During use, the high-pressure conductive fluid from lumen 704 exits through ablative port 210. The radio frequency electrode 706 may transfer energy to the conductive fluid.

As an exemplary method of use, the ablative catheter system 200 may be used for ablating a renal nerve through a blood vessel lumen, which may facilitate in treatment of conditions related to congestive heart failure.

In the exemplary method of use, referring to FIGS. 3A and 3B, an operator may insert the ablative catheter system 200 within a renal artery 300 by making an incision to the renal artery 300. Initially, while insertion, the expandable member 208 may be in its collapsed state to facilitate easy insertion and maneuvering of the ablative catheter system 200 within the lumen of the renal artery 300.

The operator may then maneuver the catheter 106 to the target site and deploy the expandable member 208 to its expanded position in the lumen of the renal artery 300. The side lobes 208c, 208d, and 208e of the expandable member 208 may contact the artery wall 302 and position the ablative port 210 at the desired distance from the artery wall 302 proximate the targeted nerve.

The operator may then actuate the controller to initiate the flow of saline out of the ablative port 210 to the artery wall 302 (FIG. 3A). The operator may then activate the radio frequency electrode 218 at the distal end of the electrical conductor 104, which conducts radio frequency electrical current to heat the saline. The saline acts as a current medium between radio frequency electrode 218 and the artery wall 302. In addition, the electrode patches 110 forms an electrical circuit between the radio frequency electrode 218 and the artery wall 302 by grounding the patient's body.

The radio frequency electrical current flows through the artery wall 302. The radio frequency electrical current generates thermal energy within the tissue at the artery wall 302 resulting in nerve ablation. In addition, the distance between the ablation port 210 and the artery wall 302 prevents irreparable damage to the tissue that may be caused by coming in direct contact with radio frequency electrode 218.

Further, the saline may escape from the distal and proximal ends of the channel 304 and dissolves into the blood stream after ablation. This process may provide a fluid cooling mechanism to dissipate the excess thermal energy generated by the ablation process into the blood stream. This process may prevent any inadvertent damage that may be caused to the adjoining tissue due to the dissipated thermal energy.

This ablation mechanism may ablate the nerve tissue in the area of the renal wall 302 that lies within the channel 304. As shown in FIGS. 4A, 4B, 4C, and 4D, the operator may rotate the catheter 106 within the renal artery 300 and repeat the ablation process to ablate nerve tissue about the complete artery wall 302. After ablating the desired region of the artery wall 302, the operator may deflate the expandable member 208 and retract the ablative catheter system 200. In some treatments, all of the nerves running along the renal artery wall are ablated at one or more locations. In other treatments, 70%, 80%, 90% or more of the nerves running along the renal artery wall are ablated to provide effective therapy.

Those skilled in the art will recognize that the present disclosure may be manifested in a variety of forms other than the specific embodiments described and contemplated herein. Accordingly, departure in form a and detail may be made without departing from the scope and spirit of the present disclosure as described in the appended claims.

What is claimed is:

1. An ablative catheter for nerve modulation, the catheter comprising:
a catheter having a proximal end, a distal end, and a first lumen extending at least partially between the proximal and distal ends, the catheter including at least one ablative port located adjacent to the distal end of the catheter;
an ablative mechanism capable of ablation using an conductive fluid circulated to the ablative port through the first lumen; and
an expandable member disposed at an outer surface of a distal portion of the catheter, the expandable member is capable of shifting between a collapsed position and an expanded position using an expansion fluid circulated to the expandable member through a second lumen,
wherein in the expanded position, the expandable member is sized and shaped to position the ablative port at a suitable distance from a wall of the vessel.

2. The ablative catheter of claim 1, wherein the ablative mechanism comprises:
an electrically conductive fluid capable of transferring radio frequency current from a current source to a target area.

3. The ablative catheter of claim 2, wherein the ablative mechanism is capable of injecting saline as the electrically conductive fluid to transfer radio frequency current to the wall of the vessel.

4. The ablative catheter of claim 1, wherein the first lumen extends between a fluid source and the ablative port.

5. The ablative catheter of claim 1, wherein the expandable member is a balloon.

6. The ablative catheter of claim 1, wherein the expandable member has a generally cartioid profile in the expanded position.

7. The ablative catheter of claim 1, wherein the expandable member is expanded by an actuating means.

8. The ablative catheter of claim 1, wherein the second lumen extends between a fluid source and an opening in the expandable member.

9. The ablative catheter of claim 1, wherein the second lumen extends between the ablative port and an opening in the expandable member.

10. The ablative catheter of claim 1, wherein the expandable member is an asymmetrically shaped member.

11. The ablative catheter of claim 1, wherein the catheter is rotatable about its elongate axis.

12. The ablative catheter of claim 1, wherein the ablative mechanism comprises an electrode at the ablative port.

13. The ablative catheter of claim 12, wherein the electrode surrounds the ablative port and has a central opening through which the conductive fluid may be ejected.

14. A renal ablation system for nerve modulation through a wall of a blood vessel, the system comprising:
an elongate member having a proximal end, a distal end, and at least one ablative port located proximate to the distal end of the elongate member;
an ablative mechanism capable of ablation using saline circulated to the ablative port; and
an expandable member disposed at an outer surface of a distal portion of the catheter, the expandable member is capable of shifting between a collapsed position and an expanded position using an expansion fluid circulated to the expandable member through a second lumen,
wherein in the expanded position, the expandable member is sized and shaped to position the ablative port is at a distance from the wall of the vessel.

15. The renal ablation system of claim 14, wherein the ablative mechanism is capable of injecting the saline with radio frequency electric current to the wall of the vessel.

16. The renal ablation system of claim 14, wherein the expandable member is a balloon.

17. The renal ablation system of claim 14, wherein the expandable member is an asymmetrically shaped member suitable to space the ablative port from a vessel wall when in the expanded position.

18. The renal ablation system of claim 14, wherein the ablative mechanism comprises an electrode at the ablative port.

19. The renal ablation system of claim 14, wherein the expandable member is fluidly connected to the ablative port.

20. A method for ablating a nerve perivascularly through a vessel lumen, the method comprising:
advancing an ablative catheter intravascularly proximate a desired location in the vessel lumen, the ablative catheter having a proximal end, a distal end, and an ablative port disposed at a location proximate the distal end of the ablative catheter, and expandable member disposed at an outer surface of a distal portion of the ablative catheter;
deploying the expandable member in an expanded position in the vessel lumen such that portions of the expandable member contact a wall of the vessel lumen and the ablative port is maintained a distance from the wall of the vessel lumen; and
activating the ablative port to inject saline with radio frequency electric current for ablating at least a portion of the nerve.
